(19) United States
(12) Patent Application Publication
(10) Pub. No.: US 2017/0239471 A1
(43) Pub. Date: Aug. 24, 2017

(54) Topical Nerve Stimulator and Sensor for Pain Management

(71) Applicant: Neurostim Solutions LLC, Cambridge, MA (US)

(72) Inventors: Graham Creasey, Menlo Park, CA (US); Hoo-Min D. Toong, Cambridge, MA (US)

(21) Appl. No.: 15/048,779

(22) Filed: Feb. 19, 2016

(51) Int. Cl. A61N 1/36; A61N 1/04

(52) U.S. Cl. CPC A61N 1/36021; A61N 1/0456; A61N 1/0492

(57) ABSTRACT

What is provided is a method and apparatus for blocking a pain signal by selectively electrically stimulating a mammalian nerve comprising: applying a dermal patch having an integral electrode in proximity to an A-alpha or A-beta touch sensitive nerve; determining a stimulation corresponding to the A-alpha or A-beta touch sensitive nerve, by logic of the dermal patch; applying the stimulation by the electrodes and a stimulator integral to the dermal patch to produce an electric field; and selectively activating the A-alpha or A-Beta touch sensitive nerve by the electric field.

### RELATED APPLICATIONS AND CLAIM OF PRIORITY

This application claims priority to and the benefit of the filing date of U.S. provisional patent application Ser. No. 62/119,134 filed Feb. 21, 2015. This application also claims priority to and the benefit of the filing date as a continuation-in-part application of U.S. utility patent application Ser. No. 14/893,946 filed Nov. 25, 2015, which claims priority to and the benefit of the filing date as a national stage application of PCT patent application serial no. PCT/US14/40240, filed May 30, 2014, which in turn claims priority to U.S. provisional patent application Ser. No. 61/828,981, filed May 30, 2013.

### TECHNICAL PROBLEM

Mammalian and human nerves control organs and muscles. Artificially stimulating the nerves elicits desired organ and muscle responses. Accessing the nerves to selectively control these responses from outside the body, without invasive implants or needles penetrating the dermis, muscle or fat tissue is desired.

A Topical Nerve Stimulator and Sensor (TNSS) device described in the related United States Patent Application Serial No. PCT/US 14/40240 filed May 30, 2014 is used to stimulate nerves. A TNSS may apply electrode generated electric field(s) in a low frequency to dermis in the proximity of a nerve. The TNSS also includes hardware and logic for high frequency (GHz) communication to mobile devices.

A wireless system including a TNSS device is described herein. Its components, features and performance characteristics are set forth in the following technical description. Advantages of a wireless TNSS system over existing transcutaneous electrical nerve stimulation devices are:

fine control of all stimulation parameters from a remote device such as a smartphone, either directly by the user or by stored programs;

multiple electrodes of a TNSS can form an array to shape an electric field in the tissues;

multiple TNSS devices can form an array to shape an electric field in the tissues;

multiple TNSS devices can stimulate multiple structures, coordinated by a smartphone;

selective stimulation of nerves and other structures at different locations and depths in a volume of tissue;

mechanical, acoustic or optical stimulation in addition to electrical stimulation;

transmitting antenna of TNSS device can focus beam of electromagnetic energy within tissues in short bursts to activate nerves directly without implanted devices;

inclusion of multiple sensors of multiple modalities, including but not limited to position, orientation, force, distance, acceleration, pressure, temperature, voltage, light and other electromagnetic radiation, sound, ions or chemical compounds, making it possible to sense electrical activities of muscles (EMG, EKG), mechanical effects of muscle contraction, chemical composition of body fluids, location or dimensions or shape of an organ or tissue by transmission and receiving of ultrasound;

TNSS devices are expected to have service lifetimes of days to weeks and their disposability places less demand on power sources and battery requirements;

combination of stimulation with feedback from artificial or natural sensors for closed loop control of muscle contraction and force, position or orientation of parts of the body, pressure within organs, and concentrations of ions and chemical compounds in the tissues;

multiple TNSS devices can form a network with each other, with remote controllers, with other devices, with the internet and with other users;

collection of large amounts of data from one or many TNSS devices and one or many users regarding sensing and stimulation, collected and stored locally or through the internet;

analysis of large amounts of data to detect patterns of sensing and stimulation, apply machine learning, and improve algorithms and functions;

creation of databases and knowledge bases of value;

convenience;
absence of wires to become entangled in clothing
showerproof and sweat proof
low profile, flexible, camouflaged or skin colored
integrated power, communications, sensing and stimulating
inexpensive
disposable TNSS, consumable electronics

power management that utilizes both hardware and software functions will be critical to the convenience factor and widespread deployment of TNSS device.

### BRIEF DESCRIPTION OF THE DRAWINGS

FIG. 1 is a depiction of a neuron activating a muscle by electrical impulse;

FIG. 2 is a representation of the electrical potential activation time of an electrical impulse in a nerve;

FIG. 3 is a graph showing pulses applied to the skin;

FIG. 4 is a graph showing symmetrical and asymmetrical pulses applied to the skin;

FIG. 5 is a cross-sectional diagram showing a field in underlying tissue produced by application of two electrodes to the skin;

FIG. 6 is a cross-sectional diagram showing a field in underlying tissue produced by application of two electrodes to the skin, with two layers of tissue of different electrical resistivity;

FIG. 7 is a cross-sectional diagram showing a field in underlying tissue when the stimulating pulse is turned off;

FIG. 8 shows potential applications of electrical stimulation to the body;

FIG. 9A is a system diagram of an example software and hardware components showing an example of a Topical Nerve Stimulator/Sensor (TNSS) interpreting a data stream from a control device;

FIG. 9B is a flow chart showing an example of a function of a master control program;

FIG. 10 is a block diagram of an example TNSS component configuration including a system on a chip (SOC);

FIG. 11 is a graph and spinal nerve diagram showing perception of pain;

FIG. 12 shows pain pathways from peripheral nerves ascending in the central nervous system to make connections to the midbrain, the brainstem and the higher centers of the brain;

FIG. 13 shows a control point for action potentials that lead to the perception of pain (nociceptive messages) near the dorsal root of the spinal cord where the A-delta and C fibers enter the spinal cord; and

FIG. 14 is a system diagram showing an example TNSS system.

### DESCRIPTION OF ACTION POTENTIALS AND NERVE PHYSIOLOGY

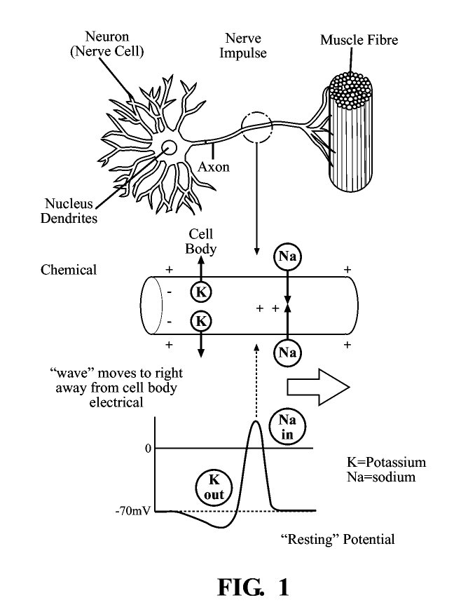

Referring to FIG. 1, a nerve cell normally has a voltage across the cell membrane of 70 millivolts with the interior of the cell at a negative voltage with respect to the exterior of the cell. This is known as the resting potential and it is normally maintained by metabolic reactions which maintain different concentrations of electrical ions in the inside of the cell compared to the outside. Ions can be actively “pumped” across the cell membrane through ion channels in the membrane that are selective for different types of ion, such as sodium and potassium. The channels are voltage sensitive and can be opened or closed depending on the voltage across the membrane. An electric field produced within the tissues by a stimulator can change the normal resting voltage across the membrane, either increasing or decreasing the voltage from its resting voltage.

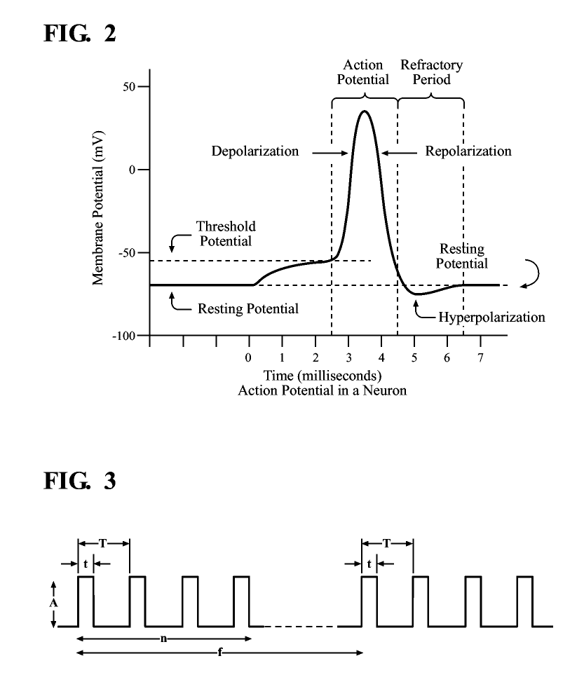

Referring to FIG. 2, a decrease in voltage across the cell membrane to about 55 millivolts opens certain ion channels, allowing ions to flow through the membrane in a self-catalyzing but self-limited process which results in a transient decrease of the trans membrane potential to zero, and even positive, known as depolarization followed by a rapid restoration of the resting potential as a result of active pumping of ions across the membrane to restore the resting situation which is known as repolarization. This transient change of voltage is known as an action potential and it typically spreads over the entire surface of the cell. If the shape of the cell is such that it has a long extension known as an axon, the action potential spreads along the length of the axon. Axons that have insulating myelin sheaths propagate action potentials at much higher speeds than those axons without myelin sheaths or with damaged myelin sheaths.

If the action potential reaches a junction, known as a synapse, with another nerve cell, the transient change in membrane voltage results in the release of chemicals known as neuro-transmitters that can initiate an action potential in the other cell. This provides a means of rapid electrical communication between cells, analogous to passing a digital pulse from one cell to another.

If the action potential reaches a synapse with a muscle cell it can initiate an action potential that spreads over the surface of the muscle cell. This voltage change across the membrane of the muscle cell opens ion channels in the membrane that allow ions such as sodium, potassium and calcium to flow across the membrane, and can result in contraction of the muscle cell.

Increasing the voltage across the membrane of a cell below −70 millivolts is known as hyper-polarization and reduces the probability of an action potential being generated in the cell. This can be useful for reducing nerve activity and thereby reducing unwanted symptoms such as pain and spasticity

The voltage across the membrane of a cell can be changed by creating an electric field in the tissues with a stimulator. It is important to note that action potentials are created within the mammalian nervous system by the brain, the sensory nervous system or other internal means. These action potentials travel along the body's nerve “highways”. The TNSS creates an action potential through an externally applied electric field from outside the body. This is very different than how action potentials are naturally created within the body.

Electric Fields that Can Cause Action Potentials

Referring to FIG. 2, electric fields capable of causing action potentials can be generated by electronic stimulators connected to electrodes that are implanted surgically in close proximity to the target nerves. To avoid the many issues associated with implanted devices, it is desirable to generate the required electric fields by electronic devices located on the surface of the skin. Such devices typically use square wave pulse trains of the form shown in FIG. 3. Such devices may be used instead of implants and/or with implants such as reflectors, conductors, refractors, or markers for tagging target nerves and the like, so as to shape electric fields to enhance nerve targeting and/or selectivity.

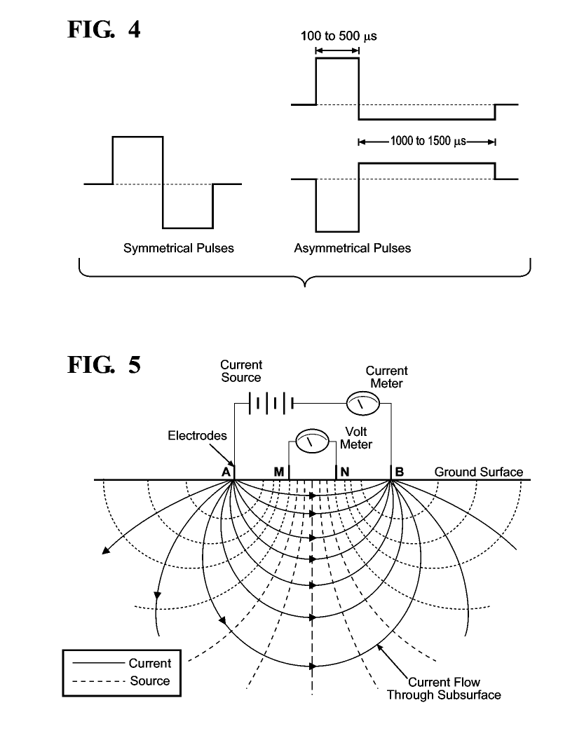

Referring to FIG. 3, the amplitude of the pulses, A, applied to the skin, may vary between 1 and 100 Volts, pulse width, t, between 100 microseconds and 10 milliseconds, duty cycle (t/T) between 0.1% and 50%, the frequency of the pulses within a group between 1 and 100/sec, and the number of pulses per group, n, between 1 and several hundred. Typically, pulses applied to the skin will have an amplitude of up to 60 volts, a pulse width of 250 microseconds and a frequency of 20 per second, resulting in a duty cycle of 0.5%. In some cases balanced-charge biphasic pulses will be used to avoid net current flow. Referring to FIG. 4, these pulses may be symmetrical, with the shape of the first part of the pulse similar to that of the second part of the pulse, or asymmetrical, in which the second part of the pulse has lower amplitude and a longer pulse width in order to avoid canceling the stimulatory effect of the first part of the pulse.

### Formation of Electric Fields by Stimulators

The location and magnitude of the electric potential applied to the tissues by electrodes provides a method of shaping the electrical field. For example, applying two electrodes to the skin, one at a positive electrical potential with respect to the other, can produce a field in the underlying tissues such as that shown in the cross-sectional diagram, FIG. 5.

The diagram in FIG. 5 assumes homogeneous tissue. The voltage gradient is highest close to the electrodes and lower at a distance from the electrodes. Nerves are more likely to be activated close to the electrodes than at a distance. For a given voltage gradient, nerves of large diameter are more likely to be activated than nerves of smaller diameter. Nerves whose long axis is aligned with the voltage gradient are more likely to be activated than nerves whose long axis is at right angles to the voltage gradient.

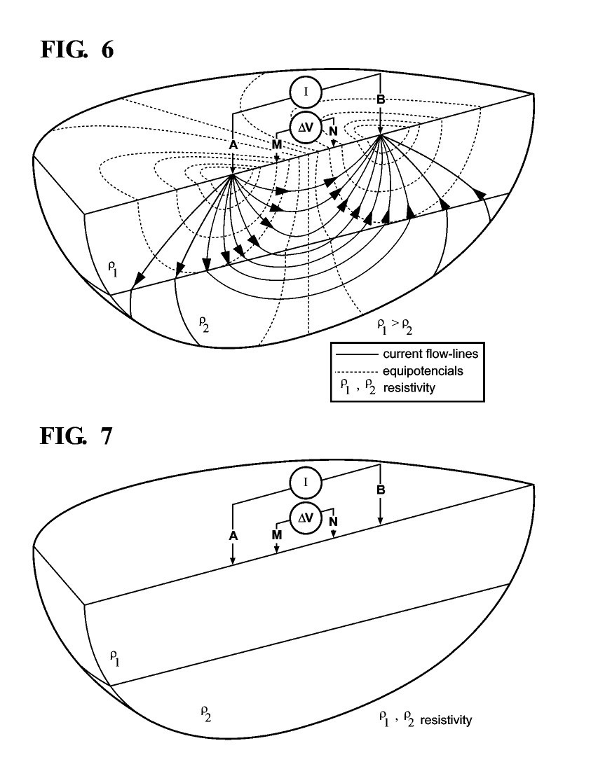

Referring to FIG. 6, applying similar electrodes to a part of the body in which there are two layers of tissue of different electrical resistivity, such as fat and muscle, can produce a field such as that shown in FIG. 6. Layers of different tissue may act to refract and direct energy waves and be used for beam aiming and steering. An individual's tissue parameters may be measured and used to characterize the appropriate energy stimulation for a selected nerve.

Referring to FIG. 7, when the stimulating pulse is turned off the electric field will collapse and the fields will be absent as shown.

It is the change in electric field that will cause an action potential to be created in a nerve cell, provided sufficient voltage and the correct orientation of the electric field occurs. More complex three-dimensional arrangements of tissues with different electrical properties can result in more complex three-dimensional electric fields, particularly since tissues have different electrical properties and these properties are different along the length of a tissue and across it, as shown in Table 1.

TABLE 1

| Electrical | | |
|---|---|---|
| Conductivity | | |
| (siemens/m) | Direction | Average |
| Blood | | .65 |
| Bone | Along | .17 |
| Bone | Mixed | .095 |
| Fat | | .05 |
| Muscle | Along | .127 |
| Muscle | Across | .45 |
| Muscle | Mixed | .286 |
| Skin (Dry) | | .000125 |
| Skin (Wet) | | .00121 |

### Modification of Electric Fields by Tissue

An important factor in the formation of electric fields used to create action potentials in nerve cells is the medium through which the electric fields must penetrate. For the human body this medium consists of various types of tissue including bone, fat, muscle, and skin. Each of these tissues possesses different electrical resistivity or conductivity and different capacitance and these properties are anisotropic. They are not uniform in all directions within the tissues. For example, an axon has lower electrical resistivity along its axis than perpendicular to its axis. The wide range of conductivities is shown in Table 1. The three-dimensional structure and resistivity of the tissues will therefore affect the orientation and magnitude of the electric field at any given point in the body.

### Modification of Electric Fields by Multiple Electrodes

Applying a larger number of electrodes to the skin can also produce more complex three-dimensional electrical fields that can be shaped by the location of the electrodes and the potential applied to each of them. Referring to FIG. 3, the pulse trains can differ from one another indicated by A, t/T, n, and f as well as have different phase relationships between the pulse trains. For example with an 8×8 array of electrodes, combinations of electrodes can be utilized ranging from simple dipoles, to quadripoles, to linear arrangements, to approximately circular configurations, to produce desired electric fields within tissues.

Applying multiple electrodes to a part of the body with complex tissue geometry will thus result in an electric field of a complex shape. The interaction of electrode arrangement and tissue geometry can be modeled using Finite Element Modeling, which is a mathematical method of dividing the tissues into many small elements in order to calculate the shape of a complex electric field. This can be used to design an electric field of a desired shape and orientation to a particular nerve.

High frequency techniques known for modifying an electric field, such as the relation between phases of a beam, cancelling and reinforcing by using phase shifts, may not apply to application of electric fields by TNSSs because they use low frequencies. Instead, the present system uses beam selection to move or shift or shape an electric field, also described as field steering or field shaping, by activating different electrodes, such as from an array of electrodes, to move the field. Selecting different combinations of electrodes from an array may result in beam or field steering. A particular combination of electrodes may shape a beam and/or change the direction of a beam by steering. This may shape the electric field to reach a target nerve selected for stimulation.

### Activation of Nerves by Electric Fields

Usually in the past selectivity in activating nerves has required electrodes to be implanted surgically on or near nerves. Using electrodes on the surface of the skin to focus activation selectively on nerves deep in the tissues has many advantages. These include avoidance of surgery, avoidance of the cost of developing complex implants and gaining regulatory approval for them, and avoidance of the risks of long-term implants.

The features of the electric field that determine whether a nerve will be activated to produce an action potential can be modeled mathematically by the Activating Function described by Rattay (Rattay F. The basic mechanism for the electrical stimulation of the nervous system. Neuroscience Vol. 89, No. 2, pp. 335-346, 1999). The electric field can produce a voltage, or extracellular potential, within the tissues that varies along the length of a nerve. If the voltage is proportional to distance along the nerve, the first order spatial derivative will be constant and the second order spatial derivative will be zero. If the voltage is not proportional to distance along the nerve, the first order spatial derivative will not be constant and the second order spatial derivative will not be zero. The Activating Function is proportional to the second-order spatial derivative of the extracellular potential along the nerve. If it is sufficiently greater than zero at a given point it predicts whether the electric field will produce an action potential in the nerve at that point. This prediction may be input to a nerve signature.

In practice this means that electric fields that are varying sufficiently greatly in space or time can produce action potentials in nerves. These action potentials are also most likely to be produced where the orientation of the nerves to the fields change, either because the nerve or the field changes direction. The direction of the nerve can be determined from anatomical studies and imaging studies such as MRI scans. The direction of the field can be determined by the positions and configurations of electrodes and the voltages applied to them, together with the electrical properties of the tissues.

As a result it is possible to activate certain nerves at certain tissue locations selectively while not activating others.

To accurately control an organ or muscle, the nerve to be activated must be accurately selected. This selectivity may be improved by using the system described herein, and described herein as a nerve signature, in several ways, as follows:

Improved algorithms to control the effects when a nerve is stimulated, for example, by measuring the resulting electrical or mechanical activity of muscles and feeding back this information to modify the stimulation and measuring the effects again. Repeated iterations of this process can result in optimizing the selectivity of the stimulation, either by classical closed loop control or by machine learning techniques such as pattern recognition and artificial intelligence;

Improving nerve selectivity by labeling or tagging nerves chemically; for example, introduction of genes into some nerves to render them responsive to light or other electromagnetic radiation can result in the ability to activate these nerves and not others when light or electromagnetic radiation is applied from outside the body;

Improving nerve selectivity by the use of electrical conductors to focus an electric field on a nerve; these conductors might be implanted, but could be passive and much simpler than the active implantable medical devices currently used;

Improving nerve selectivity by the use of reflectors or refractors, either outside or inside the body, to focus a beam of electromagnetic radiation on a nerve. If these reflectors or refractors are implanted, they may be passive and much simpler than the active implantable medical devices currently used;

Improving nerve selectivity by the use of feedback from the person upon whom the stimulation is being performed; this may be an action taken by the person in response to a physical indication such as a muscle activation or a feeling from one or more nerve activations;

Improving nerve selectivity by the use of feedback from sensors associated with the TNSS, or separately from other sensors, that monitor electrical activity associated with the stimulation; and

Improving nerve selectivity by the combination of feedback from both the person or sensors and the TNSS that may be used to create a unique profile of the user's nerve physiology for selected nerve stimulation.

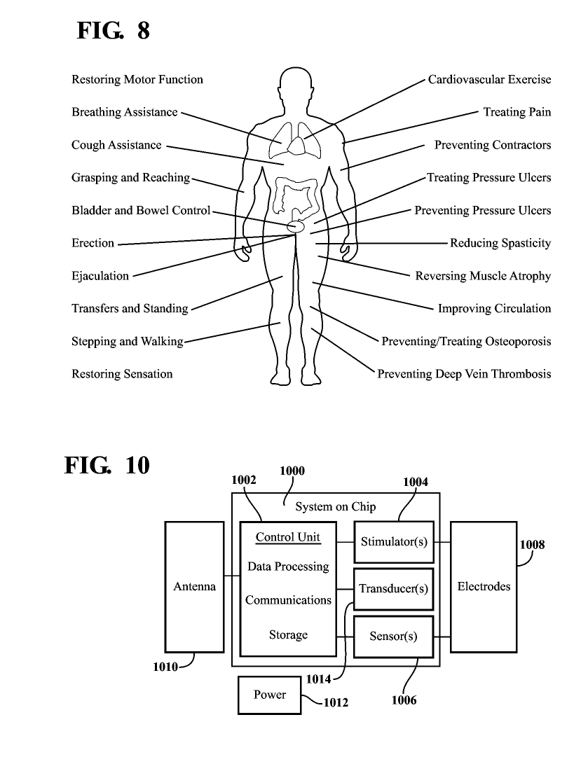

Potential applications of electrical stimulation to the body are shown in FIG. 8.

### Logic Components

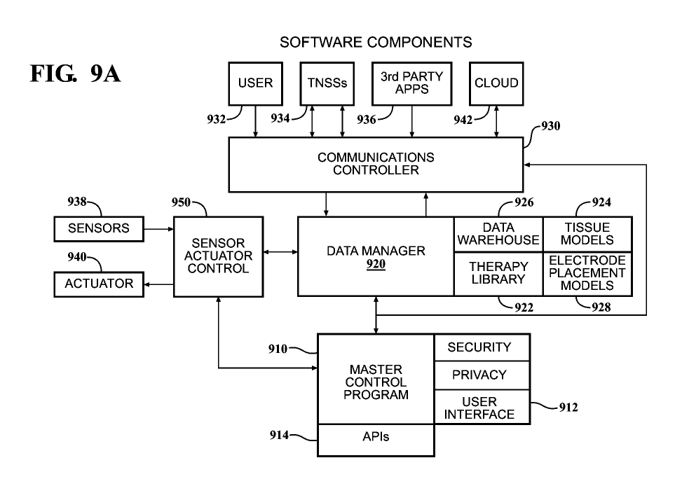

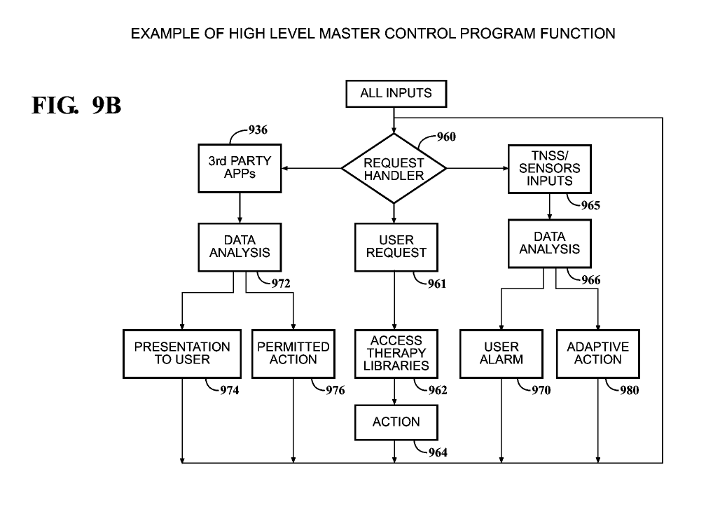

Referring to FIG. 9A, the TNSS 934 human and mammalian interface and its method of operation and supporting system are managed by a Master Control Program (MCP) 910 represented in function format as block diagrams. It provides the logic for the nerve stimulator system.

### Master Control Program

The primary responsibility of the MCP 910 is to coordinate the activities and communications among the various control programs, the Data Manager 920, the User 932, and the external ecosystem and to execute the appropriate response algorithms in each situation. The MCP 910 accomplishes electric field shaping and/or beam steering by providing an electrode activation pattern to the TNSS device 934 to selectively stimulate a target nerve. For example, upon notification by the Communications Controller 930 of an external event or request, the MCP 910 is responsible for executing the appropriate response, working with the Data Manager 920 to formulate the correct response and actions. It integrates data from various sources such as Sensors 938 and external inputs such as TNSS devices 934, and applies the correct security and privacy policies, such as encryption and HIPAA required protocols. It will also manage the User Interface (UI) 912 and the various Application Program Interfaces (APIs) 914 that provide access to external programs.

The MCP is also responsible for effectively managing power consumption by the TNSS device through a combination of software algorithms and hardware components that may include, among other things: computing, communications, and stimulating electronics, antenna, electrodes, sensors, and power sources in the form of conventional or printed batteries.

### Communications Controller

The communications controller is responsible for receiving inputs from the User 932, from a plurality of TNSS devices 934, and from 3rd party apps 936 via communications sources such as Internet or cellular networks. The format of such inputs will vary by source and must be received, consolidated, possibly reformatted, and packaged for the Data Manager 920.

User inputs may consist of simple requests for activation of TNSS devices 934 to status and information concerning the User's 932 situation or needs. TNSS devices 934 will provide signaling data that may consist of voltage readings, TNSS 934 status data, responses to control program inquiries, and other signals. The Communications Controller 930 is also responsible for sending data and control requests to the plurality of TNSS devices 934a. 3rd party applications 936 can send data, requests, or instructions for the Master Control Program 910 or User 932 via Internet or cellular networks. The Communications Controller 930 is also responsible for communications via the cloud where various software applications reside.

### Data Manager

The Data Manager (DM) 920 has primary responsibility for the storage and movement of data to and from the Communications Controller 930, Sensors 938, Actuators 940, and the Master Control Program 910. The DM 920 has the capability to analyze and correlate any of the data under its control. It provides logic to select and activate nerves. Examples of such operations upon the data include: statistical analysis and trend identification; machine learning algorithms; signature analysis and pattern recognition, correlations among the data within the Data Warehouse 926, the Therapy Library 922, the Tissue Models 924, and the Electrode Placement Models 928, and other operations. There are several components to the data that is under its control as described in the following paragraphs.

The Data Warehouse (DW) 926 is where incoming data is stored; examples of this data can be real-time measurements from TNSS devices 934 or from Sensors (938), data streams from the Internet, or control and instructional data from various sources. The DM 920 will analyze data, as specified above, that is held in the DW 926 and cause actions, including the export of data, under MCP 910 control. Certain decision making processes implemented by the DM 920 will identify data patterns both in time, frequency, and spatial domains and store them as signatures for reference by other programs. Techniques like EMG, even multi-electrode EMG, gather a lot of data that is the sum of hundreds to thousands of individual motor units and the normal procedure is to perform complex decomposition analysis on the total signal to attempt to tease out individual motor units and their behavior. The DM 920 will perform big data analysis over the total signal and recognize patterns that relate to specific actions or even individual nerves or motor units. This analysis can be performed over data gathered in time from an individual, or over a population of TNSS Users.

The Therapy Library 922 contains various control regimens for the TNSS devices 934. Regimens specify the parameters and patterns of pulses to be applied by the TNSS devices 934. The width and amplitude of individual pulses may be specified to stimulate nerve axons of a particular size selectively without stimulating nerve axons of other sizes. The frequency of pulses applied may be specified to modulate some reflexes selectively without modulating other reflexes. There are preset regimens that may be loaded from the Cloud 942 or 3rd party apps 936. The regimens may be static read-only as well as adaptive with read-write capabilities so they can be modified in real-time responding to control signals or feedback signals or software updates. Referring to FIG. 3 one such embodiment of a regimen has parameters A=40 volts, t=500 microseconds, T=1 millisecond, n=100 pulses per group, and f=20 per second. Other embodiments of regimens will vary the parameters within ranges previously specified.

The Tissue Models 924 are specific to the electrical properties of particular body locations where TNSS devices 934 may be placed. As noted previously, electric fields for production of action potentials will be affected by the different electrical properties of the various tissues that they encounter. Tissue Models 924 are combined with regimens from the Therapy Library 922 and Electrode Placement Models 928 to produce desired actions. Tissue Models 924 may be developed by MRI, Ultrasound or other imaging or measurement of tissue of a body or particular part of a body. This may be accomplished for a particular User 932 and/or based upon a body norm. One such example embodiment of a desired action is the use of a Tissue Model 924 together with a particular Electrode Placement Model 928 to determine how to focus the electric field from electrodes on the surface of the body on a specific deep location corresponding to the pudendal nerve in order to stimulate that nerve selectively to reduce incontinence of urine. Other example embodiments of desired actions may occur when a Tissue Model 924 in combination with regimens from the Therapy Library 22 and Electrode Placement Models 928 produce an electric field that stimulates a sacral nerve. Many other embodiments of desired actions follow for the stimulation of other nerves.

Electrode Placement Models 928 specify electrode configurations that the TNSS devices 934 may apply and activate in particular locations of the body. For example, a TNSS device 934 may have multiple electrodes and the Electrode Placement Model 928 specifies where these electrodes should be placed on the body and which of these electrodes should be active in order to stimulate a specific structure selectively without stimulating other structures, or to focus an electric field on a deep structure. An example embodiment of an electrode configuration is a 4 by 4 set of electrodes within a larger array of multiple electrodes, such as an 8 by 8 array. This 4 by 4 set of electrodes may be specified anywhere within the larger array such as the upper right corner of the 8 by 8 array. Other example embodiments of electrode configurations may be circular electrodes that may even consist of concentric circular electrodes. The TNSS device 934 may contain a wide range of multiple electrodes of which the Electrode Placement Models 928 will specify which subset will be activated. These Electrode Placement Models 928 complement the regimens in the Therapy Library 922 and the Tissue Models 924 and are used together with these other data components to control the electric fields and their interactions with nerves, muscles, tissues and other organs. Other examples may include TNSS devices 934 having merely one or two electrodes, such as but not limited to those utilizing a closed circuit.

### Sensor/Actuator Control

Independent sensors 938 and actuators 940 can be part of the TNSS system. Its functions can complement the electrical stimulation and electrical feedback that the TNSS devices 934 provide. An example of such a sensor 938 and actuator 940 include, but are not limited to, an ultrasonic actuator and an ultrasonic receiver that can provide real-time image data of nerves, muscles, bones, and other tissues. Other examples include electrical sensors that detect signals from stimulated tissues or muscles. The Sensor/Actuator Control module 950 provides the ability to control both the actuation and pickup of such signals, all under control of the MCP 910.

### Application Program Interfaces

The MCP 910 is also responsible for supervising the various Application Program Interfaces (APIs) that will be made available for 3rd party developers. There may exist more than one API 914 depending upon the specific developer audience to be enabled. For example many statistical focused apps will desire access to the Data Warehouse 926 and its cumulative store of data recorded from TNSS 934 and User 932 inputs. Another group of healthcare professionals may desire access to the Therapy Library 922 and Tissue Models 924 to construct better regimens for addressing specific diseases or disabilities. In each case a different specific API 914 may be appropriate.

The MCP 910 is responsible for many software functions of the TNSS system including system maintenance, debugging and troubleshooting functions, resource and device management, data preparation, analysis, and communications to external devices or programs that exist on the smart phone or in the cloud, and other functions. However, one of its primary functions is to serve as a global request handler taking inputs from devices handled by the Communications Controller 930, external requests from the Sensor Control Actuator Module (950), and 3rd party requests 936.

Examples of High Level Master Control Program (MCP) functions are set forth in the following paragraphs.

The manner in which the MCP handles these requests is shown in FIG. 9B. The Request Handler (RH) 960 accepts inputs from the User 932, TNSS devices 934, 3rd party apps 936, sensors 938 and other sources. It determines the type of request and dispatches the appropriate response as set forth in the following paragraphs.

User Request: The RH 960 will determine which of the plurality of User Requests 961 is present such as: activation; display status, deactivation, or data input, e.g. specific User condition. Shown in FIG. 9B is the RH's 960 response to an activation request. As shown in block 962, RH 960 will access the Therapy Library 922 and cause the appropriate regimen to be sent to the correct TNSS 934 for execution, as shown at block 964 labeled “Action.”

TNSS/Sensor Inputs: The RH 960 will perform data analysis over TNSS 934 or Sensor inputs 965. As shown at block 966, it employs data analysis, which may include techniques ranging from DSP decision making processes, image processing algorithms, statistical analysis and other algorithms to analyze the inputs. In FIG. 9B two such analysis results are shown; conditions which cause a User Alarm 970 to be generated and conditions which create an Adaptive Action 980 such as causing a control feedback loop for specific TNSS 934 functions, which of course can iteratively generate further TNSS 934 or Sensor inputs 965 in a closed feedback loop.

3rd Party Apps: Applications can communicate with the MCP 910, both sending and receiving communications. A typical communication would be to send informational data or commands to a TNSS 934. The RH 960 will analyze the incoming application data, as shown at block 972. FIG. 9B shows two such actions that result. One action, shown at block 974 would be the presentation of the application data, possibly reformatted, to the User 932 through the MCP User Interface 912. Another result would be to perform a User 932 permitted action, as shown at 976, such as requesting a regimen from the Therapy Library 922.

Referring to FIG. 10, an example TNSS is shown. The TNSS has one or more electronic circuits or chips 1000 that perform the functions of: communications with the controller, nerve stimulation via one or more electrodes 1008 that produce a wide range of electric field(s) according to treatment regimen, one or more antennae 1010 that may also serve as electrodes and communication pathways, and a wide range of sensors 1006 such as, but not limited to, mechanical motion and pressure, temperature, humidity, chemical and positioning sensors. In another example, TNSS interfaces to transducers 1014 to transmit signals to the tissue or to receive signals from the tissue.

One arrangement is to integrate a wide variety of these functions into an SOC, system on chip 1000. Within this is shown a control unit 1002 for data processing, communications, transducer interface and storage and one or more stimulators 1004 and sensors 1006 that are connected to electrodes 1008. An antenna 1010 is incorporated for external communications by the control unit. Also present is an internal power supply 1012, which may be, for example, a battery. An external power supply is another variation of the chip configuration. It may be necessary to include more than one chip to accommodate a wide range of voltages for data processing and stimulation. Electronic circuits and chips will communicate with each other via conductive tracks within the device capable of transferring data and/or power.

The TNSS interprets a data stream from the control device, such as that shown in FIG. 9A, to separate out message headers and delimiters from control instructions. In one arrangement, control instructions contain information such as voltage level and pulse pattern. The TNSS activates the stimulator 1004 to generate a stimulation signal to the electrodes 1008 placed on the tissue according to the control instructions. In another arrangement the TNSS activates a transducer 1014 to send a signal to the tissue. In another embodiment, control instructions cause information such as voltage level and pulse pattern to be retrieved from a library stored in the TNSS.

TNSS receives sensory signals from the tissue and translates them to a data stream that is recognized by the control device, such as the example in FIG. 9A. Sensory signals include electrical, mechanical, acoustic, optical and chemical signals among others. Sensory signals come to the TNSS through the electrodes 1008 or from other inputs originating from mechanical, acoustic, optical, or chemical transducers. For example, an electrical signal from the tissue is introduced to the TNSS through the electrodes 1008, is converted from an analog signal to a digital signal and then inserted into a data stream that is sent through the antenna 1010 to the control device. In another example an acoustic signal is received by a transducer 1014 in the TNSS, converted from an analog signal to a digital signal and then inserted into a data stream that is sent through the antenna 1010 to the control device. In certain cases sensory signals from the tissue are directly interfaced to the control device for processing.

### Application to Pain Control

### Perception of Pain

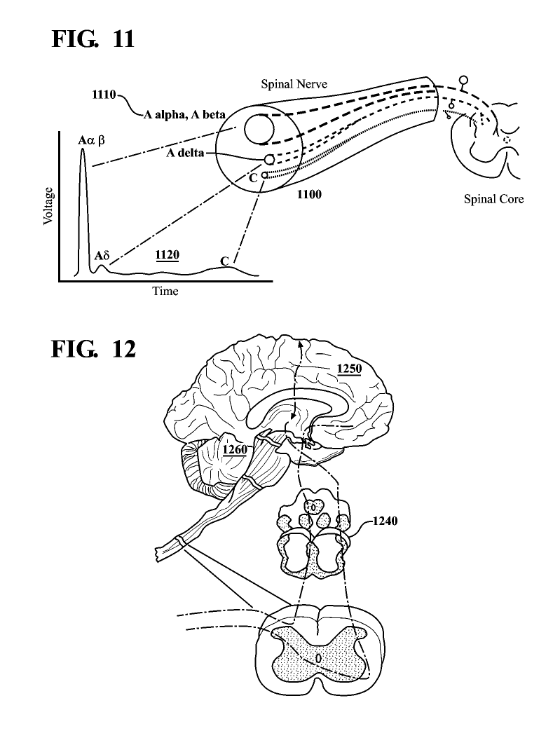

Referring to FIG. 11, pain is perceived when various types of mechanical, chemical, thermal, electrical or other stimuli initiate action potentials in small diameter non-myelinated “C” nerve fibers and slightly larger diameter myelinated “A-delta” nerve fibers 1100. The much larger A-alpha and A-beta large diameter fibers 1110 transmit touch and position information.

C fibers account for about 70% of all pain fibers. A-delta fibers conduct sharp acute pain faster than the more diffuse dull pain signals from C fibers. The signals from A-delta fibers are responsible for the withdrawal reflex that occurs within milliseconds from the initial stimulation. Because of the relative differences in conduction speeds between the myelinated A-delta and non-myelinated C fibers, the arrival of action potentials at the central nervous system upon pain stimulation occurs at different times with C fiber activation, occurring well after A-delta action potentials which in turn arrive after A-alpha and A-beta action potentials.

Referring to FIG. 12, pain pathways from the peripheral nerves described above ascend in the central nervous system to make connections to both the midbrain 1240, the brainstem and the higher centers of the brain 1250, all of which can return efferent signals downwards to various levels of the spinal cord where they interact with inhibitory interneurons and can produce analgesia or diffuse inhibition of the ascending pain signals.

The complex interactions of all these pain stimulation and inhibitory signals explain to some degree why the perception of pain is very subjective. An example of such an interaction occurs with stress-induced analgesia where wounded or injured individuals feel no pain while a battle or game is occurring but do feel the pain afterwards when they are out of the stressful situation. This effect is most likely due to the actions of descending systems of the midbrain applying both opioid and non-opioid mechanisms, as well as norepinephrine neurotransmitters and brain structures such as the amygdala and the periaqueductal grey matter.

### Treatment of Pain

The perception of pain can be reduced or abolished by initiating action potentials in larger diameter A-alpha and A-beta nerves 1110 (FIG. 11) that will modulate or block the perception of pain transmitted through smaller diameter A-delta and C nerves 1100. For example, the larger nerves can be stimulated mechanically by rubbing a portion of the body surface over the injury.

One model for the modulation of pain signals in this way is based upon the Melzack-Wall gate control theory of pain. The control mechanisms for pain signals can be viewed as a series of filters, or gates, whose closing is controlled by the cortex, the midbrain, the medulla, or other structures using many of the mechanisms described previously.

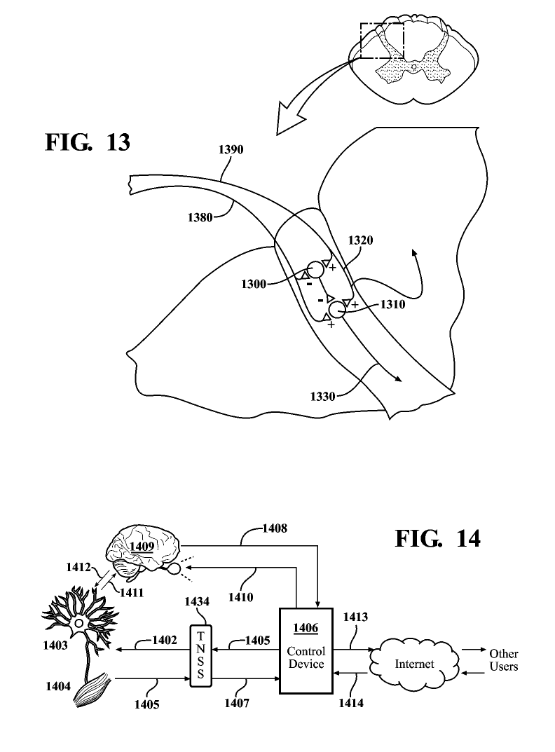

Referring to FIG. 13, the first control point for action potentials that lead to the perception of pain (nociceptive messages) is near the dorsal root of the spinal cord where the A-delta and C fibers enter the spinal cord 1380. Action potentials from mechanoreceptors that sense touch and pressure (non-nociceptive messages) carried by A-alpha and A-beta nerves also enter the spinal cord in these roots 1390.

Both nociceptive 1380 and non-nociceptive signals 1390 converge upon interneurons 1300 and upon projection neurons 1310, which in turn project axons 1330 into the spinothalamic tract ascending pathway. The most important difference between the connections made by the nociceptive and non-nociceptive nerves to the interneurons 1300 is that the nociceptive A-delta, C fibers are inhibitory and the non-nociceptive A-alpha, A-beta fibers are excitatory 1320 to the interneuron 1300. This is the circuit that forms the “gate” for segmental control of pain.

The operation of the gate proceeds as follows. Under normal conditions the inhibitory interneuron 1300 spontaneously produces action potentials that cause the projection neuron 1310 not to trigger. However, when a nociceptive fiber is activated by a pain stimulus it inhibits the interneuron 1300 from producing action potentials, which increase the chance that the projection neuron will produce action potentials, and it simultaneously stimulates the projection neuron to produce action potentials along the spinothalamic tract on the ascending pathway up the spinal cord to the brain. This results in pain signals being transmitted by the projection neuron.

However, by activating the A-alpha, A-beta touch sensitive nerves 1390, the interneuron 1300 is stimulated to produce action potentials which in turn inhibit the projection neuron 1310 from sending action potentials on the spinothalamic tract, thus “closing the gate”. The non-nociceptive fibers also supply some excitatory stimulation to the projection neuron, but on balance the action potentials produced by the interneuron generally inhibit the projection neuron from firing. By continued stimulation of the large axons, the interneuron 1300 will produce a strong hyperpolarization of the projection neuron 1310, thus reducing the chance that the projection neuron will emit action potentials.

### Electrical Treatment of Pain

The larger A-alpha and A-beta fibers can be stimulated electrically to block pain signals traveling in the A-delta and C fibers. The activation thresholds for A-alpha and A-beta fibers are much lower than the activation thresholds for A-delta and C fibers. It is thus possible to activate large A-alpha and A-beta fibers without activating A-delta and C fibers, and this can result in a reduced perception of pain signals that may be traveling in A-delta and C fibers.

Transcutaneous electrical nerve stimulation (TENS) devices produce some local analgesia by stimulating the A-alpha, A-beta fibers in the skin.

TNSS devices also have the ability to stimulate nerves deep in the body by focusing electric fields within the tissues using electrodes on the surface of the skin. This expands the possibilities of reducing pain beyond current use of transcutaneous electrical nerve stimulation TENS devices, which primarily stimulate surface non-nociceptive fibers. Specific patterns of electrical stimulation coupled with techniques of field steering, beam forming and beam steering may be used to stimulate nerves deep within the tissues. For example, the ability of the TNSS to focus electrical stimulation on nerves deep in soft tissues around the lumbar spine may be useful in reducing low back pain and spasm resulting from disc disease, arthritis and other degenerative conditions of the spine. It may also be useful in many other conditions causing pain where non-invasive and non-medication-based control of pain is desired.

The TNSS may in some cases stimulate sensory nerves in the peripheral nervous system that would lead to indirectly activating other parts of the central nervous system or descending neural pathways that provide inhibitory efferent signals from the higher centers of the brain, the midbrain, brainstem, and the spinal cord.

### How the TNSS System Works for Pain

### Simple Stimulation

This is a representative sequence of events to illustrate the use of the TNSS system:

1: Referring to FIG. 14, User feels in the brain 1409 the perception of pain as a result of signals 1411 from the peripheral nervous system.

2: User activates the TNSS 1401 via a button on the TNSS or a Control Device 1406 which may be a smartphone or a dedicated device. A dedicated device is a small portable device resembling a key fob and containing electronic circuits for storage and operation of programs and buttons that the user can operate. When the user presses a button on the Control Device this can cause it to transmit radio-frequency signals 1408 to the TNSS to control the operation of the TNSS. The dedicated device can also receive radio-frequency signals 1407 from the TNSS. The TNSS and the Control Device are under software control, responding to an action from the user. There will be safeguards to prevent false activations or unnecessary repetitive activations.

3: The activation by the user causes a stimulator in the TNSS to send electrical stimulation signals 1402 to the appropriate nerve(s) 1403 that block the pain signals thereby relieving the pain as discussed previously. The TNSS can stimulate the appropriate nerve(s) to relieve pain with a preset pulse signal, or the user can select from variety of pulse signals, and their intensities; this might be implemented as one or more of a plurality of ‘buttons’ on the interface of a smartphone or actual buttons on a dedicated device. The user selects from programs to deal with mild pain, moderate pain, or severe pain; the programs may provide an intermittent or a continuous pulse signal and the signal may have a timeout of a duration chosen by the user.

4: The user can reactivate the TNSS 1401 either immediately if the pain is not completely abated, or the next time he/she feels the sensation of pain, or can maintain activation for a desired period.

### Complex Stimulation

This includes logging functions, incorporating data from the cloud, and data from other sensors and sources. This is a representative sequence of events to illustrate some of the additional functions in addition to the Simple Stimulation described above.

Upon activation of the TNSS one or more of the following functions can occur:

The User's activation profile is recorded by the TNSS 1401 and shared with the Control Device 1306 or transmitted over the internet 1413. The activation profile consists of a User ID, stimulation signal identifier, date and time of day, and if the user interface permits, user activity at the time of activation Historical data can be gathered and analyzed for the user's benefit.

Data from the Internet 1414 may be accepted by the Control Device 1406 and/or the TNSS 1401; types of data may be instructions from a healthcare professional, population data (e.g. messages from support group members), statistical analyses and trend data (relative to the individual user or across populations). This data can be passed through to the user, or cause actions to be taken (e.g. alarms, notifications, etc.).

Data 1405 can be gathered from other sensors, from other sensors, which may be located in the TNSS 1401, in the control device, and other patches, or in other devices, on a continuous basis or only when the TNSS 1401 is activated. When the TNSS 1401 is activated, these data can be used to alter or modify the stimulation signals 1402 that the TNSS 1401 transmits to the user. An example would be an acoustic transceiver, for example implemented in MEMS that can both transmit and receive acoustic data to create acoustic images of the structure beneath the electrodes or elsewhere in the body that may be affected by the neural stimulation. This would allow the TNSS to gather image data of the neurally stimulated region and aid in locating and identifying structures to be stimulated, providing information on which to base field or beam forming and steering functions of the TNSS 1401.

### Adaptive Stimulation

To assist the user in locating and stimulating the appropriate large nerves, the TNSS 1401 is capable of steering an electric field or electric beam within the tissues using a plurality of electrodes. This includes the ability to steer the electric field or beam to activate nerves deep in the body selectively, without activating adjacent nerves or nerves nearer the surface. The user is able to control this steering directly, or to allow the device to steer stimulation to a variety of locations. Based on perception of pain relief, the user identifies and selects the pattern and location of stimulation that provides optimal pain relief. These patterns and locations are presented by the control device to the user 1410.

This mode of operation will make use of the various feedback loops inherent in the TNSS System as shown in FIG. 14.

The normal activation signal 1408 to cause the TNSS 1401 to be activated comes from the user's perception that pain needs to be alleviated and action to activate the Control Device 1406 as described previously. There is a plurality of other methods that can control the source of pain in parallel or separately from this method.

With an acoustic transceiver, the TNSS 1401 can send an acoustic pulse and derive an image of the stimulated region to identify the locations of deep nerves. The TNSS can then focus electrical stimulation to activate a deep nerve selectively.

The device may also be able to sense other effects of stimulating nerves, such as movement of contracting muscle 1404, or the electrical signals produced by contracting muscle, to assist in identifying nerves being stimulated. The device may thus be able to select patterns and locations of stimulation with little or no intervention by the user.

1. A method of blocking a pain signal by selectively electrically stimulating a mammalian nerve comprising:
applying a dermal patch having an integral electrode in proximity to an A-alpha or A-beta nerve;
determining a stimulation corresponding to the A-alpha or A-beta nerve, by logic of the dermal patch;
applying the stimulation by the electrodes and a stimulator integral to the dermal patch to produce an electric field; and
selectively activating the A-alpha or A-Beta nerve by the electric field.

2. The method of claim 1, further comprising stimulating an A-alpha or an A-beta fiber without stimulating an A-delta or a C fiber.

3. The method of claim 1, further comprising stimulating an A-alpha or an A-beta fiber to block a pain signal traveling in an A-delta or a C fiber.

4. The method of claim 1, further comprising causing an interneuron to produce an action potential by the stimulation.

5. The method of claim 1, further comprising inhibiting the projection neuron from transmitting a pain signal, by the stimulation.

6. The method of claim 1, further comprising inhibiting a pain projection neuron from producing an action potential along a spinothalamic tract on an ascending pathway up a spinal cord to a brain, by the stimulation.

7. The method of claim 1, further comprising stimulating an interneuron by the activation of the A-alpha or A-beta nerve to produce an action potential that inhibits a projection neuron from sending an action potential on a spinothalamic tract.

8. The method of claim 1, further comprising inhibiting a projection neuron from firing by stimulating the A-alpha or A-beta nerve.

9. The method of claim 1, further comprising:
activating the electrodes to produce a desired electric field by a stimulator integral to the dermal patch a plurality of times;
producing a hyperpolarization of a projection neuron by activating an interneuron by the stimulation; and
reducing emission by the projection neuron of action potentials by the stimulation.

10. The method of claim 1, further comprising receiving a manual command to activate the stimulator.

11. The method of claim 1, further comprising receiving a manual command to activate the stimulator from a remote control device.

12. The method of claim 1, further comprising stimulating the selected A-alpha or A-beta nerve with a preset pulse.

13. The method of claim 1 further comprising providing a plurality of stimulation pulses having different intensities or durations.

14. The method of claim 1 further comprising providing a pulse program of stimulation pulses including mild pain, moderate pain, and severe pain programs.

15. The method of claim 1 further comprising providing intermittent or continuous stimulation pulses.

16. The method of claim 1 further comprising providing a library of stimulation pulses, the pulses having pre-set intensities or durations.

17. An apparatus for blocking a pain signal by selectively electrically stimulating a mammalian nerve comprising:
a dermal patch having an integral electrode configured to select an A-alpha or A-beta nerve;
logic to determine a stimulation corresponding to the A-alpha or A-beta nerve to produce an electric field; and
a stimulator to activate the electrodes in proximity to the A-alpha or A-beta nerve to selectively activate the A-alpha or A-Beta nerve by the electric field.

18. The apparatus of claim 17 further comprising a library of stimulation pulses, the pulses having pre-set intensities or durations.
